(19) United States
(12) Patent Application Publication
(10) Pub. No.: US 2019/0014824 A1
(43) Pub. Date: Jan. 17, 2019

(54) PORTABLE DEVICE AND METHOD FOR VAPORIZING MATERIALS

(71) Applicant: Torque Research and Development, Inc., San Diego, CA (US)

(72) Inventors: Daniel Yazbeck, San Diego, CA (US); Joshua Londano, San Diego, CA (US); James Panther, Carlsbad, CA (US); Jason Panther, San Diego, CA (US)

(21) Appl. No.: 15/819,707

(22) Filed: Nov. 21, 2017

Related U.S. Application Data
(60) Provisional application No. 62/533,355, filed on Jul. 17, 2017.

(51) Int. Cl. A24F 47/00; G05B 15/02; A61M 21/02; G06F 21/31

(52) U.S. Cl. CPC A24F 47/008; G05B 15/02; A61M 21/02; A61M 2021/0066; A61M 2205/3334; A61M 2205/50; G06F 21/31

(57) ABSTRACT

A portable device, and more particularly a portable device for vaporizing materials such as oils for inhalation. Also, methods for vaporizing materials and methods of operating portable vaporizing devices. In addition, networks for providing operational features and/or instructions for operation of vaporizing devices and for monitoring and collecting operational data. In preferred embodiments, a vaporizing device and/or methods for operating a vaporizing device wherein the vaporizing device includes one or more of the following features: security features to regulate operation of the device; automatic detection of vaporizing material identity; user experience monitoring and data collection; and/or temperature adjustment of vaporizing material based on detected material for vaporization.

### CROSS-REFERENCE TO RELATED APPLICATIONS

This application claims priority to U.S. Provisional Application No. 62/533,355 filed on Jul. 17, 2017, entitled “Portable Device for Vaporizing Materials and Methods Related Thereto”, the entire disclosure of which is hereby incorporated by reference.

The following U.S. Patents are also hereby incorporated by reference: U.S. Pat. Nos. 7,359,802; 7,189,353; 7,144,553; 6,759,010; 6,610,367; 6,170,318; 6,093,308; 6,013,229; 6,010,616; 5,959,191; 5,951,846; 5,911,872; 5,891,398; 5,788,833; 5,698,089; 5,571,401; 8,394,330; 7,966,132; 7,955,561; 7,595,023; 7,175,885; 7,122,152; 6,962,675; 6,773,926; 6,631,333; 6,571,603; 6,455,319; 6,387,329; 6,350,369; 6,290,911; 7,359,802; 7,189,353; 7,144,553; 6,759,010; 6,610,367; 6,170,318; 6,093,308; 6,013,229; 6,010,616; 5,959,191; 5,951,846; 5,911,872; 5,891,398; 5,788,833; 5,698,089; 5,571,401; 7,966,132; 6,571,603; and 6,350,369.

### FIELD OF THE INVENTION

The present invention generally relates to a portable device, and more particularly relates to a portable device for vaporizing materials such as oils for inhalation. This invention also relates to method of vaporizing materials and methods of operating portable vaporizing devices.

### BACKGROUND OF THE INVENTION

Vaporization is the process of changing liquid or solid substances to a gas. Devices used to vaporize active ingredients in materials such as tobacco, cannabis, or other similar materials are known as vaporizers. Generally, vaporizers such as electronic cigarettes or electronic smoking devices are products that allow a user to inhale aerosol containing oil, wax, nicotine and other substances. Unlike conventional cigarettes, vaporizers are typically composed of a rechargeable battery-operated heating unit, a cartridge that may contain oil or other materials, and an atomizer that, when heated, converts the contents of the cartridge into an aerosol. Further, the electronic smoking device industry has evolved and now provides more products than electronic cigarettes, like hookah pens and electronic cigars. Some industry trade groups refer to these products as Personal Electronic Vaporizing Units (PEVUs).

Such vaporizing devices may be used to consume cannabis and cannabis concentrates. This practice is becoming more common as states begin to legalize cannabis for medical and recreational use. A user's experience after consuming a cannabis product varies with the chemical profile of the cannabis. Problematically for consumers, distributors, healthcare providers, and regulators alike is the fact that the chemical profile between different strains of cannabis is highly variable. Further, having PEVU's in a home where children are present may pose a risk to the health and safety of the children.

Therefore, there is a need for an apparatus to analyze the quality and chemical composition of vaporizing materials. There is a need for a vaporizer device that allows a user to monitor consumption habits, such as frequency of use, and wirelessly control access to the vaporizer device. Further, there is a need for a vaporizer device that stores a user's experience with vaporizing materials. Further there is a need for a device that allows a healthcare provider, or other third party, to monitor and control the dosages consumed while using the vaporizer device. Such a need is especially strong when a vaporizer is used to consume controlled substances, or substances requiring a medical prescription, such as cannabis.

Further limitations and disadvantages of conventional and traditional approaches will become apparent to one of skill in the art through comparison of described systems with some aspects of the present disclosure, as set forth in the remainder of the present application and with reference to the drawings.

### SUMMARY OF THE INVENTION

The present disclosure generally relates to a vaporizing device. In other embodiments, the inventions disclosed herein relate to methods of using vaporizing devices and/or methods of monitoring the ingestion of vaporizable material. In certain embodiments, the inventions relate to a communication system, including a vaporizing device for vaporizing materials while tracking consumption habits and the effects of vaporizing materials. In one embodiment, a vaporizing device for vaporizing materials is disclosed.

In at least one embodiment, there is provided: a vaporizing device for vaporizing materials comprising: a battery unit for supplying power to the apparatus; a notification unit, electrically connected to the battery unit; a circuit board, electrically connected to the battery unit and the notification unit, further comprising, a heating unit for heating the materials to a vaporizing temperature, a counter unit for recording and communicating inhalation data, electrically connected to the battery unit, and a communication unit integrated with the counter unit and communicatively connected to a computing device for transmitting inhalation data to and receiving instructions from the computing device wherein the computing device is communicatively connected to a communication network; a vaporization unit thermally connected to the heating unit and configured to store and heat the vaporization materials; and a mouthpiece having a lower end, an upper end and an inhalation passageway wherein the lower end is configured to receive the vaporization unit, the upper end is configured to contact a user's lips, and the inhalation passageway runs from the upper end to the lower end allowing air to travel freely through the device.

In at least one alternative embodiment, there is provided a method for monitoring and regulating a user's consumption of vaporizing materials comprising providing a vaporizing device including at least one flow meter and heating element associated therewith; providing at least one computer processor and at least one computer readable medium in communication with the computer processor; the computer readable medium and the computer processor being communicably connected to a network; the computer readable medium including, stored thereon, a set of computer readable and executable instructions for processing by the computer processor, the computer readable and executable instructions being defined to permit control of the parameters of vaporizer usage and being defined to permit data monitoring of the vaporizer usage; the computer readable and executable instructions also defining a vaporizer end user interface, comprised of graphical user interface components; providing one or more data input devices configured to permit one or more users to input data, in the form of transmittable data, corresponding to vaporizing device usage, for storage, for analysis, or for transmission to a monitoring entity; providing one or more display screens configured for displaying the graphical user interface and for displaying vaporizer data and; configuring the flow meter so that when the vaporizing device is operated by an end user to ingest vaporized gas, the volume of ingested vaporized gas is measured and a data point is collected and processed by the computer processor; and configuring the graphical user interface to prompt the end user to enter data pertaining to the end user's experience upon ingestion of the vaporized gas.

In yet another embodiment there is provided a method for monitoring and regulating a user's consumption of vaporizing materials comprising providing a vaporizing device including at least one flow meter and heating element associated therewith; providing at least one computer processor and at least one computer readable medium in communication with the computer processor; the computer readable medium and the computer processor being communicably connected to a network; the computer readable medium including, stored thereon, a set of computer readable and executable instructions for processing by the computer processor, the computer readable and executable instructions being defined to permit control of the parameters of vaporizer usage and being defined to permit data monitoring of the vaporizer usage; the computer readable and executable instructions also defining a vaporizer end user interface, comprised of graphical user interface components; providing one or more data input devices configured to permit one or more users to input data, in the form of transmittable data, corresponding to vaporizing device usage, for storage, for analysis, or for transmission to a monitoring entity; providing one or more display screens configured for displaying the graphical user interface and for displaying vaporizer data and; configuring the flow meter so that when the vaporizing device is operated by an end user to ingest vaporized gas, the volume of ingested vaporized gas is measured and a data point is collected and processed by the computer processor; configuring the graphical user interface to prompt the end user to enter data pertaining to the end user's experience upon ingestion of the vaporized gas; and automatically machine determining the identity of the ingestible material installed in the vaporizing device; installing a quantity of vaporizable and ingestible material at an intake portion of the vaporizing device; and automatically machine determining the identity of the vaporizable and ingestible material.

In certain non-limiting embodiments, the device is powered by a battery unit which supplies power to various other components of the device. The device includes a notification unit that generally notifies a user when the device is in operation or the power level remaining in the battery unit. Also included is a circuit board, electrically connected to the battery unit and the notification unit. Optionally integrated onto the circuit board are a heating unit, a counter unit, and a communication unit. The heating unit heats vaporization materials to a temperature sufficient to cause vaporization. The counter unit senses when and records a user's inhalation and is electrically connected to the battery unit. The communication unit is integrated with the counter unit and communicatively connected to a computing device wherein the computing device is also connected to a communication network. The device also includes a vaporization unit that is thermally connected to the heating unit and configured to store and heat vaporization materials. Lastly, the device includes a mouthpiece that is configured to contact a user's lips on one end and configured to receive the vaporization unit on the opposite end. The mouthpiece has an inhalation passageway that runs through the piece to allow air to flow freely through the device.

In another embodiment, there is provided a non-transitory computer readable storage medium contained in a computing device that stores coded instructions executable for monitoring and regulating a user's consumption of vaporizing materials while using a portable vaporizing apparatus comprising coded instructions. The coded instructions include the first step of receiving cartridge data on a single vaporization material. Next is the step of receiving inhalation data from the vaporizing device, and converting the inhalation data into a graphical format enabling a user to monitor inhalation habits over time for any single vaporization material. Lastly, in some embodiments, a step of transmitting the cartridge data and the inhalation data to one or more external devices is included.

Also disclosed is a communication system for monitoring and regulating a user's consumption of vaporizing materials while using a portable vaporizing apparatus. The system first includes a vaporizing device with the capabilities of embodiments of the device disclosed in this specification. The system further includes a computing device containing the non-transitory computer readable storage medium also disclosed herein, wherein the computing device is communicatively connected with the vaporizing device and configured to transmit instructions to the vaporizing device. Next, the system includes a database communicatively connected with the computing device wherein the database is configured to receive data from the computing device. Lastly the system includes a processing unit communicatively connected with the database and the computing device wherein the processing unit is configured to receive data from the database and to send instructions to the computing device.

Another embodiment disclosed is a method to monitor inhalation data and communicating the monitored inhalation data to a computing device over a communication network. The method includes the first step of a user inhaling a vaporizing material through the vaporizing device disclosed herein. Next, a step if performed to transmit inhalation data to a computing device communicatively connected to the vaporizing device, and optionally storing the data in a non-transitory computer readable storage medium. The method further includes storing the inhalation data in the computing device, and receiving to the computing device the user's feedback relating to how the vaporizing materials made the user feel. Further, the method includes transmitting the user's feedback to a database. Then a user may view the inhalation data from the computing device, and the computing device provides the user with information pertaining to a total number of inhalations taken over time. Lastly, the computing device, providing the user with a summary of how the vaporizing material has made the user feel over time.

In certain non-limiting embodiments, the notification unit of the vaporizing device is an LED board. In such or other embodiments, the communication unit comprises a BLE chip. In certain embodiments, a BLE ANT is electrically connected to the BLE chip, for improving the BLE chip's ability to send and receive wireless signals. In some of these embodiments, an LED driver is electrically connected to the LED board and the circuit board. In certain embodiments, the vaporizer also includes, or is connected to (directly or wirelessly/virtually), a sensor array, electrically connected to the BLE chip, and fluidly connected to the vaporizing unit, for determining a chemical composition of vaporizing materials. In certain optional embodiments, the battery unit may be recharged wirelessly.

In certain embodiments wherein measurement of ingested material is utilized, a counter unit is included, comprising a flow rate sensor. In similar or other embodiments, the counter unit comprises a pressure sensor.

In some embodiments, it is desirable to monitor the temperature used for vaporization. In certain of such embodiments, a voltage potentiometer is electrically connected to the battery unit and the heating unit, for varying the amount of power sent to the heating unit. In certain embodiments of the methods of the inventions described herein, steps are undertaken to measure vaporization dosages. In at least one of such embodiments, the method includes the following method steps: defining a vaporizing gas dosage determined as having characteristics and qualities beneficial to an end user, the dosage comprising a volume of gas having a quantity X; using the flow meter to measure a first volume of vaporized gas ingested by the end user, as the vaporized gas passes through the flow meter, and assigning the first volume of vaporized gas ingested as quantity Y₁; comparing the value of quantity X to the value of quantity Y₁ and if the value of quantity Y₁ is less than the value of quantity X, the computer processer processing the computer readable and executable instructions permits the continued operation of the vaporizing device, but if the value of quantity Y₁ is approximately equal to or greater than the value of quantity X, the computer processer processing the computer readable and executable instructions at least temporarily shuts down vaporizing functions of the vaporizing device so that the end user is prevented from ingesting further quantities of vaporized gas.

In another one of such optional embodiments, the method also includes the following method steps: using the flow meter to measure a second volume of vaporized gas ingested by the end user, as the vaporized gas passes through the flow meter, and assigning the second volume of vaporized gas ingested as quantity Y₂; the computer processer processing the computer readable and executable instructions automatically adding the value of quantity of Y₁ to the value of quantity Y₂ to obtain a sum representing the cumulative volume Y_{C} of vaporized gas ingested by an end user; comparing the value of quantity X to the value of cumulative volume Y_{C} and if the value of cumulative volume Y_{C} is less than the value of quantity X, the computer processer processing the computer readable and executable instructions permits the continued operation of the vaporizing device, but if the value of cumulative volume Y_{C} is approximately equal to or greater than the value of quantity X, the computer processer processing the computer readable and executable instructions at least temporarily shuts down vaporizing functions of the vaporizing device so that the end user is prevented from ingesting further quantities of vaporized gas. In still other optional embodiments, the following additional method steps are performed, wherein the monitoring entity, and not the end user, selects the vaporizing gas dosage having a quantity X and transmits information corresponding to the vaporizing gas dosage from a monitoring entity location to a location corresponding to the data input device and the vaporizing device; and wherein because the monitoring entity selects the vaporizing gas dosage having a quantity X, the monitoring entity maintains restrictive control of the type and amount of vaporized gas ingested by the end user.

In certain preferred but optional embodiments, the monitoring entity is a physician and the vaporizing gas dosage having a quantity X is selected and designated pursuant to a prescription prescribing a quantity of ingestible material. In at least one of these embodiments, the vaporizing gas dosage having a quantity X is transmitted to an end user patient of the licensed physician over communication network such that data corresponding to the vaporizing gas dosage having a quantity X is received by the data input device and the vaporizing device of the end user patient. Further, the computer processer processing the computer readable and executable instructions controls the operating status of the vaporizing device so that the end user is only permitted to ingest vaporizing material utilizing the vaporizing device in accordance with the prescription.

In certain optional embodiments, the method includes the steps of provisioning the set of computer readable and executable instructions for processing by the computer processor to include a security protocol and provisioning the data input device to require compliance with the security protocol prior to the computer processor activating the vaporizing device for usage by an end user.

One optional type of security protocol that may be used is a password requirement, wherein a password must be entered into the data input device utilizing the graphical user interface; and wherein upon successful entry of a correct password into the data input device, the computer processor activates the vaporizing device for usage by an end user. In another embodiment employing a security protocol, a step is included comprising provisioning the set of computer readable and executable instructions for processing by the computer processor so that the security protocol automatically deactivates the vaporizing device after a predetermined duration of non-usage of the vaporizing device is measured.

In certain particularly preferred (but still optional) embodiments, wherein a security protocol is employed, the security protocol includes the following method steps: provisioning the set of computer readable and executable instructions for processing by the computer processor to include a security protocol; provisioning the data input device to require compliance with the security protocol prior to the computer processor activating the vaporizing device for usage by an end user. In certain variations of these embodiments, the security protocol comprises automatically machine determining the identity of the ingestible material installed in the vaporizing device and the computer processor thereafter comparing the identity of the ingestible material to data contained in the prescription of the physician. If the identity of the ingestible material matches data in the prescription of the physician, the vaporizing device is activated for usage by the computer processor. If the identity of the ingestible material does not match data in the prescription of the physician, the vaporizing device remains deactivated and cannot be used to vaporize material for ingestion by an end user.

In certain embodiments, applicants have discovered that the herein disclosed apparatus and methods can be used in novel fashion in clinical drug trials. In at least one of such embodiments, the monitoring entity (elsewhere more generally described herein) is an entity collecting data for use and analysis in clinical trials; and the input data, input by users, corresponds to vaporizing device usage, and pertains to the end user's experience upon ingestion of the vaporized gas. In such embodiments, the data is collected and analyzed in connection with a clinical trial associated with the ingestion of the vaporized gas.

In certain embodiments using a graphical user interface to collect data pertaining to user experiences, the method includes the method step of prompting the end user to enter data pertaining to the end user's experience upon ingestion of the vaporized gas. Optionally, this step includes prompting the end user to enter one or more data points selected from the group consisting of: satisfaction, dissatisfaction, positive effect, negative effect, happy, sad, relaxed, agitated, energetic, inactive, social, anti-social, focused, scattered, motivated, discouraged, and no effect.

In certain embodiments, the applicants herein have determined that optimal temperatures for vaporizing materials are different depending on the material type. Also, when certain vaporizing temperatures are exceeded, the vaporized material may become harmful. Conversely, if the temperature is not high enough, the vaporizing material may not be adequately vaporized thus rendering its ingestion ineffective. Addressing and/or combating these issues, at least some embodiments of this invention further include the following method step of selecting a temperature of the heating element as a vaporizing temperature of the vaporizable and ingestible material. In certain preferred (but optional) embodiments, the process is automatic. In such an example embodiment, a computer processor compares the identity of the vaporizable and ingestible material to data stored pertaining to the vaporizable and ingestible material; and automatically machine determines the temperature of the heating element as a vaporizing temperature of the vaporizable and ingestible material, using the data stored pertaining to the vaporizable and ingestible material.

In still other optional embodiments, the step of installing the quantity of vaporizable and ingestible material at the intake portion of the vaporizing device is performed by installing a removable cartridge containing the vaporizable and ingestible material to the intake portion of the vaporizing device. In some (but not all) of these embodiments, the removable cartridge includes data information pertaining to the identity of the vaporizable and ingestible material. During operation, the vaporizing device reads the data information in order to perform the step of automatically machine determining the identity of the vaporizable and ingestible material.

Certain specific examples of the invention are now described below with respect to certain non-limiting embodiments thereof as illustrated in the following drawings wherein:

### BRIEF DESCRIPTION OF CERTAIN EXAMPLE DRAWINGS

The drawings submitted with and which form a part of this patent application each illustrate an embodiment, or one or more components of an embodiment, of a non-limiting example of Applicant's invention. While these drawings depict certain preferred embodiments of Applicants' invention, as well as certain particularly desirable features thereof, they are intended to be examples only and should not be construed to limit the scope of Applicant's invention.

FIG. 1 illustrates a general view of the device for vaporizing materials for inhalation, in accordance with at least one embodiment;

FIG. 2 illustrates a block diagram of a circuit board, in accordance with at least one embodiment; and

FIG. 3 illustrates a block diagram of a firmware framework, in accordance with at least one embodiment.

FIGS. 4a and 4b illustrate a user interface of the computing device, in accordance with at least one embodiment.

FIG. 5 illustrates an environmental diagram of the system to monitor inhalation data through a computing device over a communication network, in accordance with at least one embodiment.

FIG. 6 illustrates a flowchart of a method to monitor inhalation data and further communicating the monitored inhalation data to a computing device over a communication network, in accordance with at least one embodiment.

### DETAILED DESCRIPTION OF CERTAIN EMBODIMENTS

The present disclosure is best understood with reference to the detailed figures and description set forth herein. Various embodiments are discussed below with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment”, “at least one embodiment”, “an embodiment”, “one example”, “an example”, “for example”, and so on indicate that the embodiment(s) or example(s) may include a particular feature, structure, characteristic, property, element, or limitation, but not every embodiment or example necessarily includes that particular feature, structure, characteristic, property, element, or limitation. Furthermore, repeated use of the phrase “in an embodiment” does not necessarily refer to the same embodiment.

Unless defined otherwise, all technical and scientific terms used herein have the same meaning as commonly understood by one of ordinary skill in the art to which this invention belongs. Although any method and material similar or equivalent to those described herein can also be used in the practice or testing of the present invention, the preferred methods and materials are described. All publications, patents, and patent applications mentioned herein are incorporated in their entirety.

It is also noted that as used herein and in the appended claims, the singular forms “a”, “and”, and “the” include plural referents unless the context clearly dictates otherwise. In the claims, the terms “first”, “second”, and so forth are to be interpreted merely as ordinal designations they shall not be limited in themselves. Furthermore, the use of exclusive terminology such as “solely”, “only” and the like in connection with the recitation of any claim element is contemplated. It is also contemplated that any element indicated to be optional herein may be specifically excluded from a given claim by way of a “negative” limitation. Finally, it is contemplated that any optional feature of the inventive variation(s) described herein may be set forth and claimed independently or in combination with any one or more of the features described herein.

All references, including publications, patent applications, and patents cited herein are hereby incorporated by reference to the same extent as if each reference were individually and specifically indicated to be incorporated by reference and were set forth in its entirety herein.

The recitation of ranges of values herein are merely intended to serve as a shorthand method of referring individually to each separate value falling within the range, unless otherwise indicated herein, and each separate value is incorporated into the specification as if it were individually recited herein.

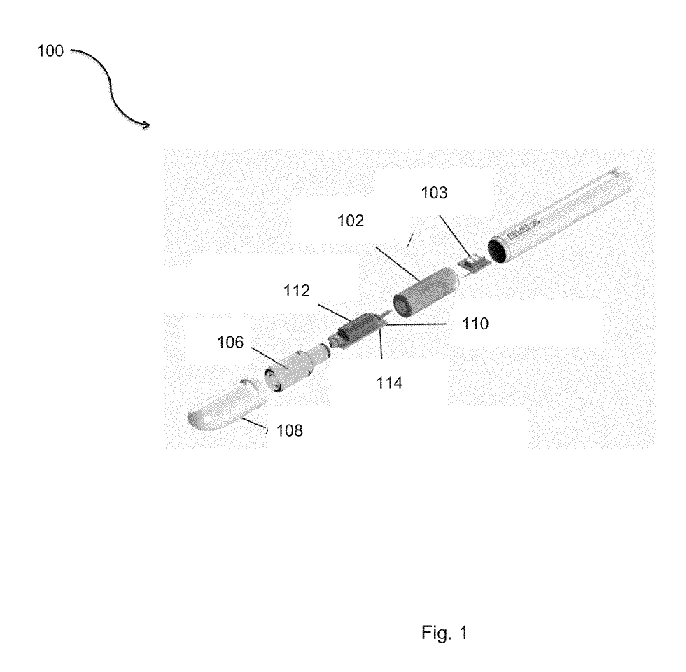

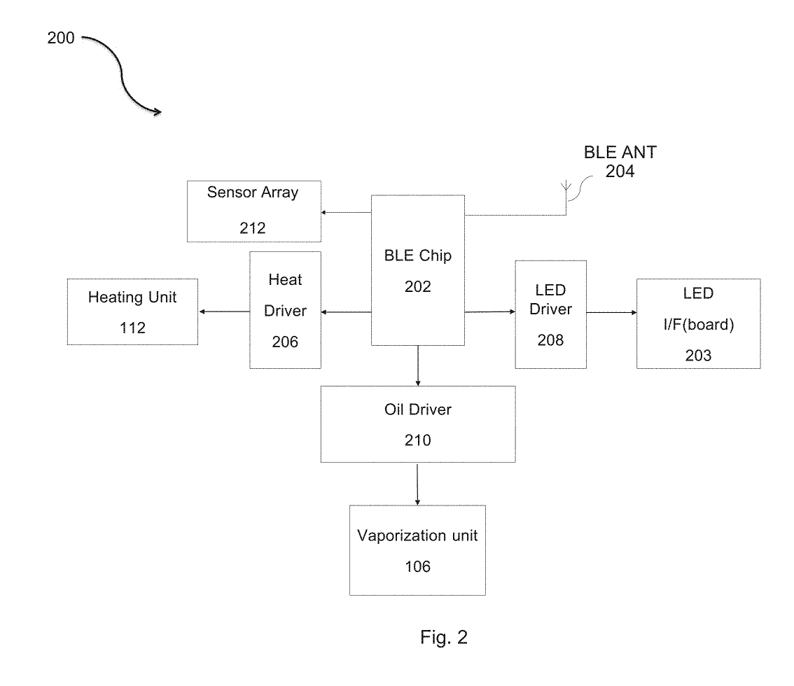

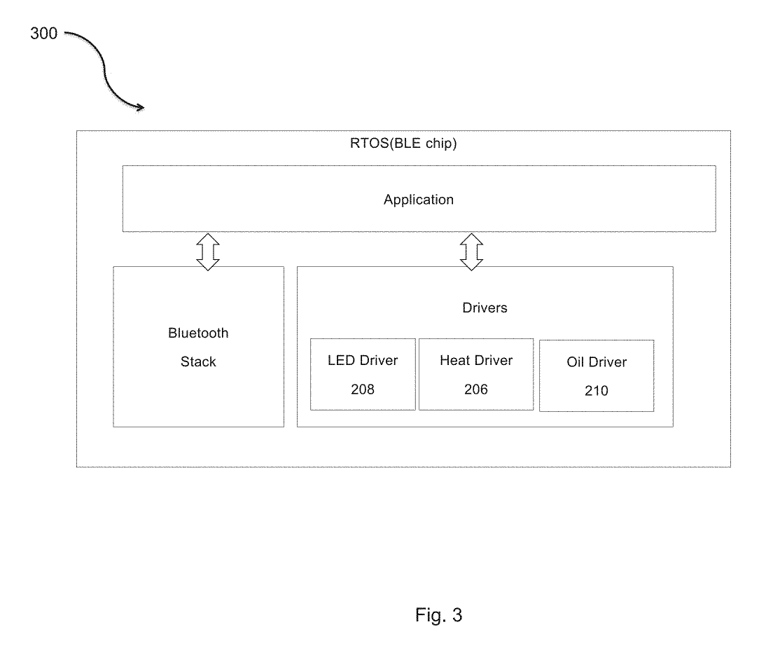

FIG. 1 illustrates a general view of the device 100, in accordance with at least one embodiment. The device 100 includes a battery unit 102, a notification unit 103, a vaporization unit 106, a mouthpiece unit 108, and a circuit board 200. The circuit board 200 (explained in FIG. 2) includes a counter unit 110, a heating unit 112, a communication unit 114, an LED driver 208, heat driver 206, an oil driver 210, and firmware 300. The LED Driver 208, heat driver 206, and oil driver 210 are discussed in FIG. 2. Firmware 300 is discussed in FIG. 3.

The battery unit 102 powers the notification unit 103, and the circuit board 200, of FIG. 2. In an embodiment, the battery unit 102 is a rechargeable Lithium-Ion Battery. The battery unit 102 may be recharged using a wire or wirelessly through a wireless charging dock. In one embodiment, the battery unit 102 has a capacity of about 1000 mAh-1200 mAh.

The notification unit 103 notifies the user regarding the status of the battery unit 102, completion/incompletion of the inhalation data transferred to a computing device through the communication unit 114, number of dosages consumed by the user, and power on/off notifications. In certain embodiments, the notification unit may include a light-emitting diode board (“LED board”), a liquid crystal display screen (“LCD screen”), an organic light-emitting diode screen (“OLED screen”), or an equivalent. In other embodiments, the notification unit includes one or more LED units, an audio unit, and/or combination thereof. In FIG. 1, the notification unit 103 is shown as an LED board, electrically connected to the battery unit 102 on a proximal end of the battery unit 102. In other embodiments, the notification unit 103 may be integrated with the circuit board 200, which also may contain a counter unit 110, a heating unit 112, and a communication unit 114.

The vaporization unit 106 stores a vaporization material. Vaporization materials may be oil, wax, herbs, cannabis, nicotine, and/or a combination thereof. Vaporization materials may come packaged in cartridges that may be loaded directly into the vaporization unit 106. Typically, vaporization materials have unique names and unique chemical profiles. Each type of vaporization material has a unique vaporization temperature at which its active ingredients begin to vaporize. The vaporization unit 106 is configured to receive and conduct heat from the heating unit 112 sufficient to heat each vaporization material to its vaporization temperature. In one embodiment, the vaporization unit 106 is configured to receive vaporization materials in the form of a cartridge.

The mouthpiece unit 108 is configured with the vaporization unit 106. The mouthpiece unit 108 provides a surface configured to receive a user's lips, so that the user can comfortably inhale from the device 100 after placing their lips on the mouthpiece unit 108. The mouthpiece unit 108 includes a passageway leading from the vaporization unit 106 to a proximal end of the vaporizer device 100. The user draws air in through the mouthpiece unit 108 to activate the heating unit 112. Then the air in the vaporization unit 106 is heated and causes the vaporization materials in the vaporization unit 106 to vaporize.

The counter unit 110 is a flow rate sensor, a pressure sensor, or an equivalent, capable of measuring and counting the number of doses, or “puffs”, a user takes from the vaporizer device 100. For example, when a user takes a “puff,” the counter unit will track the volume of gas ingested and send this information to the computing device. This specification uses “doses” and “puffs” interchangeably. When the specification refers to “consumption of vaporization materials,” it is generally referring to a user taking a dose or taking a puff using the vaporizer device 100, and thereby consuming vaporization materials via inhalation of aerosol. FIG. 1 shows the counter unit 110 integrated on the circuit board 200 with the heating unit 112, and the communication unit 114. In other embodiments, the counter unit 110 may be separate from the circuit board. In one embodiment, the counter unit 110 also operates to activate the heating unit 112 upon sensing that a user is taking a puff by sending a signal to the communication unit 114, which then activates the heating unit 112. A person of skill in the art will find beneficial the incorporation of the counter unit 110 because it enables a user to monitor their use of vaporization materials. Additionally, because the counter unit 110 is integrated with the communication unit 114, the counter unit 110 enables a third party, such as a doctor or healthcare administrator, to remotely monitor a patient's consumption of vaporization materials.

The heating unit 112 is an element capable of generating heat such as a resistive metallic heat coil or ceramic heating element. The heating unit 112 transfers heat to the vaporization unit 106 for heating the vaporization material stored in the vaporization unit 106 to a vaporization temperature. For example, in one embodiment the heating unit 112 can heat the vaporization unit 106 to temperatures of 300-400 degrees Fahrenheit. The heating unit 112 is electrically coupled to the battery unit 102, and thermally coupled with the vaporization unit 106. FIG. 1 shows the heating unit 112 as a wire coil and utilizes a male-female coupling style connection to connect to the vaporization unit 106. The heating element 112 is shown with a male connector, and couples to the female connector on a lower proximal end of the vaporization unit 106. The heating element 112 may also thermally connect with the vaporization unit 106 such as by wrapping the coil directly around the vaporization unit 106, or integrating a wire coil into the vaporization unit 106. In one embodiment, the heating unit 112 is partially contained inside the vaporization unit 106. In some embodiments, the heating unit 112 may include nichrome. Some embodiments may include a voltage potentiometer, electrically connected to the battery unit 102, for varying the amount of power sent to the heating unit 112 so a user can vary the intensity of vapor consumed in each puff. For example, the voltage potentiometer may be a slider or a thumbwheel that extend from the circuit board through the outer casing of the device, enabling a user to manipulate the voltage potentiometer by hand.

The communication unit 114 is integrated with the counter unit 110 to communicate the inhalation data such as count of the dosages, or puffs, to a computing device 302 (discussed in FIG. 3). Such a communication may be performed, according to various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), IEEE 802.11, 802.16, 2G, 3G, 4G cellular communication protocols, and/or Bluetooth Low Energy Technology (BLE) communication protocols. BLE is variation of original traditional Bluetooth standard with low power consumption. BLE is a Wireless Personal Area Network standard which operates at 2.4 GHz band. In an embodiment, a BLE chip consumes less power and goes to sleep when not in use, therefore the present device 100 can be operated using coil cell battery for longer duration. Accordingly, the communication unit 114 preferably utilizes a BLE chip. For example, in one embodiment, the communication unit 114 utilizes a BLE chip, or BLE chipset, and has a transmission range of about 4 feet. In some embodiments, the communication unit 114 includes an on-board processor.

FIG. 2 illustrates a block diagram of circuit board 200, in accordance with at least one embodiment. FIG. 2 is explained in conjunction with FIG. 1. The diagram includes a BLE chip 202, a BLE ANT 204, an LED driver 208, an LED board 203, an oil driver 210, a vaporization unit 106, a heat driver 206, and a heating unit 112.

The BLE chip 202 is an integrated circuit, or system of circuits, that supports at least the BLE protocol. BLE chip 202 may also support other wireless protocols such as ANT. The BLE chip 202 represents one embodiment of the communication unit 114, which is integrated with the counter unit 110 to communicate the inhalation data such as count of the dosages to a computing device. In one embodiment, the BLE Chip 202 has an on-board processor, such as the Nordic Semiconductor nRF52810 chip

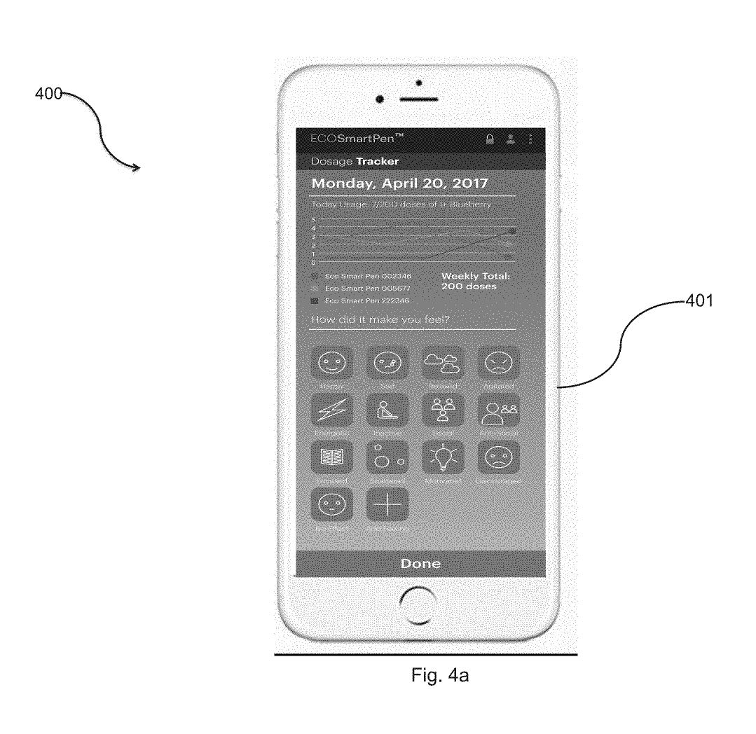

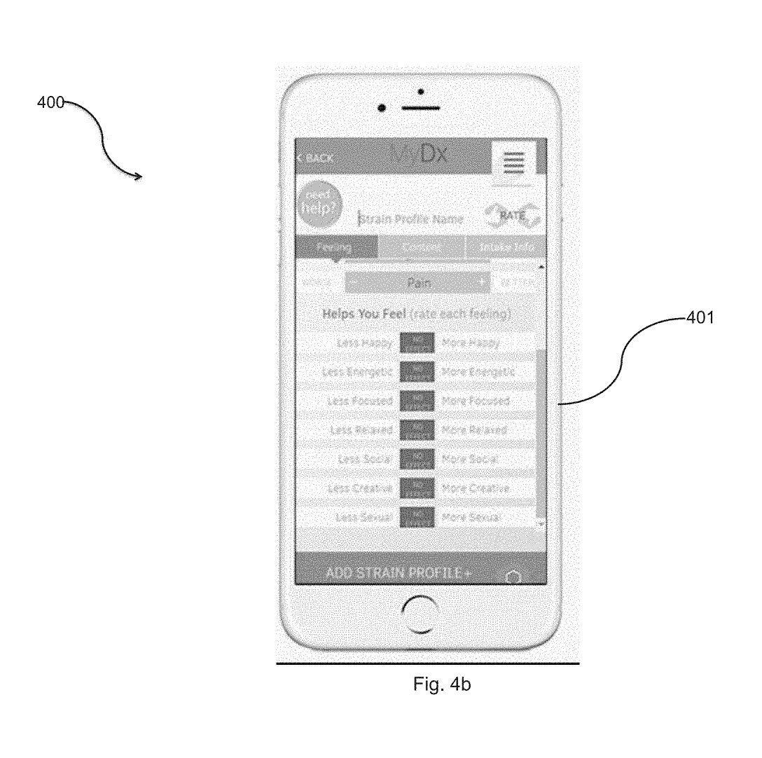

The BLE ANT 204 is a BLE antenna that communicatively couples the BLE chip 202, with the computing device 401 (discussed in FIG. 4), and is electrically connected to the BLE chip 202.

The LED driver 208 is a semiconductor that prevents LEDs from drawing too much voltage as the temperature of the LEDs increases. The LED driver 208 is electrically connected to the LED board 203 and may also be electrically connected to the battery unit 102. FIG. 1, or the circuit board 200, or both. FIG. 2 shows one embodiment where the LED driver 208 is also electrically connected to the BLE chip 202.

The LED board 203 is a unit having LEDs mounted onto a conductive substrate. These may also be referred to as surface mount LEDs. The LED board 203, shown in FIG. 2, is one embodiment of the notification unit 103 discussed previously. See discussion of the notification unit 103 for additional details. In at least one embodiment, the oil driver 210 is electrically connected to the BLE chip 202.

In certain embodiments, the circuit board 200 may also include a sensor array 212. The sensor array 212 may be, for example, the CannaDx™, or another sensor performing a similar function. The sensor array 212 is electrically connected to the BLE Chip 202, which supplies power to the sensor array 212, and fluidly connected to the vaporization unit 106. The sensor array 212 is configured to determine a chemical composition of vaporizing materials loaded into the vaporization unit 106. The sensor array 212 generally consists of an array of sensors coated with different sensor films. For example, the sensors may be coated with a polymer wherein each sensor has a varying film thickness. When the sensor film is exposed to molecules contained in vaporizing material vapors, the film may expand, changing the electrical resistance in the sensor array, allowing for identification of particular analytes present in the vapor. Certain embodiments require a user to take a puff or blow air through the mouthpiece 108 to carry vaporizing material vapor to the sensor array 212, or to take a control reading.

FIG. 3 illustrates a block diagram 300 of a firmware framework, in accordance with at least one embodiment. FIG. 3 is explained in conjunction with FIG. 2. To communicate with the various drivers 206, 208, 210, the BLE chip 202 includes real-time operating system (“RTOS”). RTOS is an operating system which has capability to suffice the real time requirements of the present device 100.

Referring now to FIGS. 4a and 4b, a non-limiting example embodiment of a user interface 400 is illustrated. The user interface 400 is executable on a computing device 401.

The computing device 401 may be, but is not limited to, a smart phone, a personal computer, a laptop, a personal digital assistant (PDA), a mobile device, a tablet, or any other similar computing device. In a preferred embodiment, the computing device 401 is a smart phone such as an iPhone.

The user interface 400 is a software application executable from the computing device 401. The vaporizer device 100, of FIG. 1, is communicatively connected with the computing device 401 via the communication unit 114 of FIG. 1, which allows the user interface 400 to receive data from the vaporizer device, such as inhalation data. The user interface 400 is operative to perform several important features such as: receiving information about vaporizing materials inserted into the vaporizing device 100, tracking a user's doses, or puffs, receiving feedback from a user regarding the user's experience with the vaporizing materials, scheduling reminders for a user to take a prescribed dose, and locking the vaporizer device 100 for safety or to prevent abuse.

The user interface 400 is operable to receive data on the particular vaporization material loaded into the vaporizer device 100, and associate inhalation and user feedback data with a particular vaporizing material. For example, different strains of cannabis commonly have unique names. In California, two popular strains of cannabis are Blue Dream and Sour Diesel. The chemical profile of Blue Dream is unique, and, accordingly, inhalation of Blue Dream will give a user a different experience than Sour Diesel. The user interface 400 allows a user to input the particular vaporization material being consumed and track data against that particular vaporization material. In one embodiment, the vaporization device 100 will automatically transmit data identifying vaporization material, to the computing device 401, upon the insertion of a compatible cartridge into the vaporization unit 106.

FIG. 4a illustrates the inhalation tracking feature of the user interface 400. Referring to FIGS. 1 and 2, the counter unit 110 senses airflow when a user takes a puff, which activates the heating unit 112 heating the vaporization materials stored in the vaporization unit 106 to a vaporizing temperature. In addition, upon sensing a puff, the counter unit 110 sends a signal to the BLE chip 202 which in turn sends a signal to the computing device 401. The computing device 401 stores inhalation data, and the user interface 400 quantifies, and organizes inhalation data, allowing a user to easily monitor their habits over time. FIG. 4a illustrates a plot of inhalation data over time, one embodiment of the inhalation tracking feature of the user interface 400. In some embodiments, the computing device 401 vibrates on receiving the inhalation data from the vaporization device 100. This inhalation tracking feature provides a great benefit to a user that wants to track a medically prescribed prescription, or otherwise monitor their use habits.

FIG. 4a also illustrates the user interface feature of receiving user feedback. FIG. 4a provides a list of example emotions a user can input into the interface such as: happy, sad, relaxed, agitated, energetic, inactive, social, anti-social, focused, scattered, motivated, discouraged, and no effect. For example, after a user takes a puff of a particular vaporization material, the user can input that the user felt relaxed. In one embodiment, a user may input into the user interface 400 how they want to feel after consuming a vaporizing material. Tracking the way a particular vaporizing material makes a user feel provides great benefit to a user because over time the user will be able to track how certain vaporization materials make the user feel. Further, the user will be able to use this information to tailor purchasing habits toward the vaporization materials best suited to the user's needs. Similarly, FIG. 4b illustrates the user interface 400 feature of receiving user input in the form of a strain profile.

Certain other embodiments of the user interface 400 are operative to turn off, or lock, the vaporizer device 100 of FIG. 1. For example, referring to FIG. 1, when the vaporizer device 100 is unlocked, a user draws air in through the mouthpiece 108, the counter unit 110 senses the airflow, which activates the heating unit 112, heating the vaporization materials stored in the vaporization unit 106 to a vaporizing temperature. However, certain embodiments of the user interface 400 enable a user to turn off, or lock, the device remotely. When locked, the heating unit 112 will not activate, even if the counter unit 110 senses an inhalation. The lockout feature is important and beneficial to users because it makes the vaporizer device safer and gives the user more control over device operation. For example, a parent may not want their children to use the device. Using the lockout feature in the user interface 400, a parent could easily prevent a child from using the vaporizer device by locking the device. Another example of an important benefit of the lockout feature is that it makes the vaporizer device safer. Previous devices may have posed a safety hazard if the device activated while in a user's pocket or purse. In the present device, a user may use the lockout feature to prevent accidental device activation when the device is not in use. In other embodiments of the user interface 400, the lockout feature may be scheduled in order to lock the vaporizer device 100 after a certain amount of time has elapsed or during specified periods of time. For example, if a parent knows that a child will be home from school each day from 3:00 PM until 6:00 PM, the parent can schedule the vaporizer device to be locked. [Question:

Other embodiments of the user interface 400 may implement the lockout feature by generating a code to activate the vaporization device 100 of FIG. 1. This feature enables the vaporization device 100 to provide access only to an authorized user and prevents children, or other unauthorized users, from using the device accidently or unknowingly. In an exemplary embodiment, the code is generated by a random code generator. Examples of the generated code include but are not limited to numerals, alphabetical, alphanumeric, symbols and/or combinations thereof. In another embodiment the code is defined by a user, so that to unlock the device 100, the user must input their own unique code. There are various advantages of the present code such as security and access control.

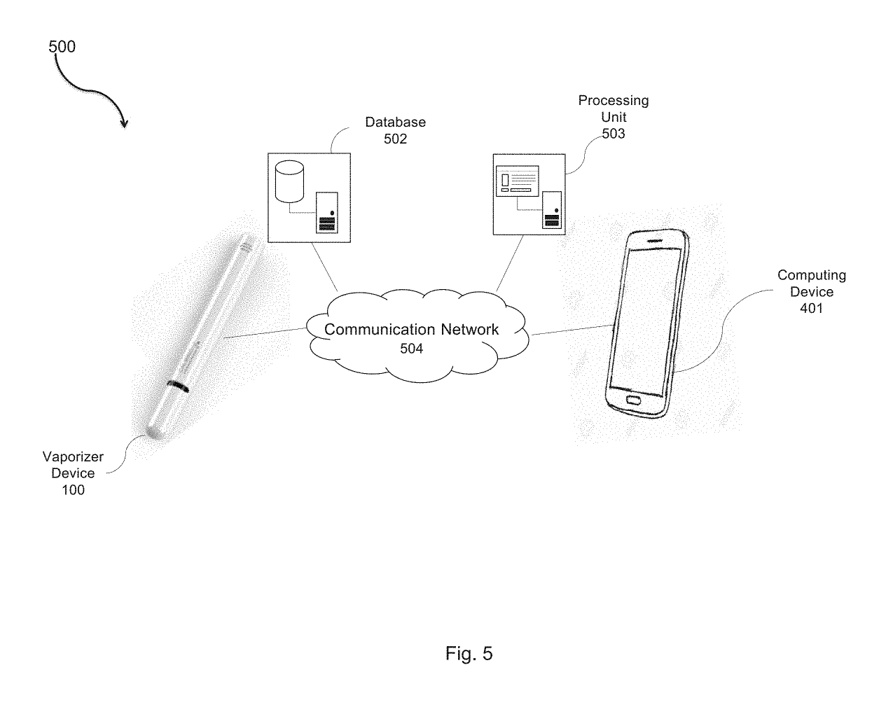

FIG. 5 illustrates an environmental diagram of a system 500 to monitor inhalation data through a computing device 401 over a communication network 504, in accordance with at least one embodiment. The system 500 includes a vaporizer device 100, a computing device 401, a database 502, a processing unit 503, and a communication network 504.

The processing unit 503 is a host computer, a server, or other central processing unit capable of interpreting and executing instructions. The processing unit 503 may be implemented based on a number of processor technologies known in the art. Examples of the processing unit 503 include, but not limited to, an X86-based processor, a Reduced Instruction Set Computing (RISC) processor, an Application-Specific Integrated Circuit (ASIC) processor, a Complex Instruction Set Computing (CISC) processor, and/or other processor.

Generally, the processing unit 503 may be configured for receiving data sent from the computing device 401, sending instructions to computing device 401, and accessing data stored in database 502. In one embodiment, the processing unit 503 refers to a computing device or a software framework hosting an application or a software service. In another embodiment, the processing unit 503 is implemented to execute procedures such as, but not limited to, programs, routines, or scripts stored in one or more memories for supporting the hosted application or the software service. In other embodiments, the hosted application or the software service is configured to perform one or more predetermined operations. The processing unit 503 may utilize various types of application servers such as, but not limited to, a Java application server, a .NET framework application server, a Based application server, a PHP framework application server, or any other application server framework.

The processing unit 503 is communicatively connected with a database 502, and the computing device 401. In one embodiment, the processing unit 503 may be capable of locking the vaporizing device 100 to prevent use or misuse. Referring to FIGS. 5 and 1, the vaporizer device 100 enables a user to draw air in through a mouthpiece unit 108, which is sensed by the counter unit 110 activating a heating unit 112. The communication unit 114, integrated with the counter unit 110, sends the inhalation data, such as count of the dosages and user experiences, to the computing device 401. The computing device 401 then sends the inhalation data to at least one of either the processing unit 503, or the database 502. The ability of the processing unit 503 to receive data directly from the vaporizer device 100 provides a great benefit to a user. For example, in the healthcare setting, a healthcare provider could use the processing unit 503 to monitor a patient's use of vaporizing materials by comparing the patient's actual dosage against a prescribed dosage. If the healthcare provider suspects the patient may be abusing vaporizing materials, the healthcare provider can use the processing unit 503 to lock the vaporizing device, and thereby prevent any further abuse.

The database 502 is a computing system capable of storing a collection of interrelated data from a plurality of users of the vaporizing device 100. The database 502 is communicatively connected to the processing unit 503, and the vaporizing computing device 401. The interrelated data stored in database 502 may include, but is not limited to: user inhalation data, user feedback data, user location data, vaporization data, vaporization material location data. The inhalation data, user feedback data, and vaporization material location data may also be associated with a particular vaporizing material to better track how certain vaporizing materials make users feel. Capabilities of the database 502 include sending data to and receiving data from the processing unit 503 and the vaporizer device 100. The database 502 may also store and send user feedback data to the computing device 401. This data may be displayed in the user interface 400 of FIGS. 4a and 4b. In one embodiment, the database 502 is configured to receive data from many users, such as a user's experience with a certain vaporizing material and where the user purchased the particular vaporizing material. The database 502 provides an important benefit to users. For example, in the healthcare setting, using the processing unit 503, a healthcare provider can access the database 502 to determine how particular vaporizing materials are making their patients feel, and determine where a patient might buy a particular vaporizing material that would best suit their needs.

In one embodiment, the processing unit 503, or the computing device 401 may instruct the database 502 to conduct a query and find vaporizing materials that produce a desired effect, located with a defined geographical region. For example, if a user wants to find vaporizing materials that result in a feeling of creativity, the database 502 can run a query, comparing user feedback for various vaporizing materials in the user's geographic area, and recommend a vaporizing material that reportedly made users feel creative.

The communication network 504 is a medium through which a vaporizer device 100, one or more computing devices 401, and the processing unit 503 communicate with each other. Like the communication unit 114 of FIG.1, the communication network 504 may communicate in accordance with various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), IEEE 802.11, 802.16, 2G, 3G, 4G cellular communication protocols, and/or Bluetooth (BT) communication protocols. The communication network 108 includes, but is not limited to, the Internet, a cloud network, a Wireless Fidelity (Wi-Fi) network, a Wireless Local Area Network (WLAN), a Local Area Network (LAN), a telephone line (POTS), and/or a Metropolitan Area Network (MAN).

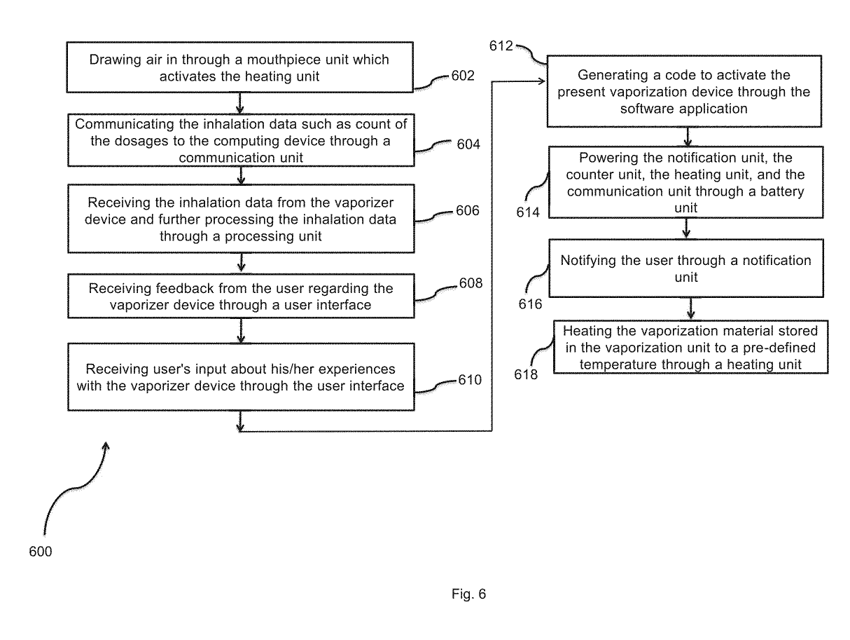

Referring now to FIG. 6, provided is flowchart 600 which illustrates one embodiment of a method to monitor inhalation data and further communicating the monitored inhalation data to a computing device over a communication network. The method initiates with the step 602 of drawing air in through a mouthpiece unit which is sensed by a counter unit, and thereby activates the heating unit. In an embodiment, the mouthpiece unit is integrated with a vaporizer device. Then the method includes the step 604 of communicating the inhalation data such as count of the dosages to the computing device through a communication unit integrated with a counter unit. In an embodiment, the vaporizer device includes a communication unit, and a counter unit. Examples of the communication unit include, but are not limited to, Bluetooth and Wi-Fi. In an exemplary embodiment, the Bluetooth has a transmission range of about 4 feet.

Next, the method includes the step 606 of receiving the inhalation data from the vaporizer device and further processing the inhalation data through a processing unit. The processed information is stored in a database configured with the computing device. The computing device includes a software application.

The method further includes the step 608 of receiving feedback from the user regarding the vaporizer device through a user interface. In an embodiment, the software application includes a user interface. Further the method includes the step 610 of receiving a user's input about his/her experiences with the vaporizer device through the user interface. Additionally, the user may enter the information pertaining to the inhalation into the user interface. The software application stores the inhalation data and user's experiences and displays through the user interface.

Furthermore, the method includes the step 612 of generating a code to activate the present vaporization device through the software application. This feature enables the vaporization device to provide the access only to the authorized user and may be used to prevent children from using the device. In an exemplary embodiment, the code is generated by a random code generator. Examples of the generated code include but are not limited to numerals, alphabetical, alphanumeric, symbols and/or combinations thereof. There are various advantages of the code generation feature such as additional security and access control.

Further the method includes the step 616 of notifying the user through a notification unit regarding the status of the battery unit, completion/incompletion of the inhalation data transferred to a computing device through the communication unit, number of the dosage consumed by the user, and power on/off notifications. In one embodiment, the notification unit includes one or more LED units, an audio unit, and/or combination thereof. In an embodiment, the LED units are connected to the battery unit and operable to indicate the battery level.

Next, the method includes the step 618 of heating the vaporization material stored in the vaporization unit to a pre-defined temperature through a heating unit electrically coupled to the battery unit. In an embodiment, the heating unit is partially contained inside the vaporization unit 106. In an exemplary embodiment, the heating unit may include nichrome.

### Non-Limiting Exemplary Embodiment

One particularly preferred embodiment is now described as a non-limiting but representative example of one of the several inventions disclosed herein.

In this example embodiment, a vaporizer user is diagnosed by a doctor, and the doctor issues a prescription for a vaporizable material which can be vaporized using one or more of the devices described herein. The prescription itself is entered into a computer system which, itself, is connected to a network. The vaporizing device of the user is also selectively connected to the network, but may be connected or disconnected using a switch (physical or software based) on the device itself or the software of an accompanying computing unit, such as an iPhone, also communicably connected to the vaporizer device.

The user purchases the prescribed material, which may be provided in cartridges for example, and distributed by legally authorized facilities. The cartridge itself preferably includes a bar code or similar identifying code or device, so that when the cartridge is installed on the vaporizing device, the vaporizing device automatically recognizes the material loaded in the cartridge by reading the bar code, or by obtaining data from an alternative material identification mechanism. To enable this purpose, the vaporizing device may have a miniature bar code reader, or similar data reading device, to read the data on the cartridge to determine the content of the cartridge.

Once the cartridge is installed, the vaporizing device reads the data on the cartridge and automatically determines the content of the cartridge. Next, the vaporizing device, when it is connected to the network, transmits a data query into the network to determine whether the material detected as contained within the installed cartridge contains the material which was prescribed by the prescribing doctor. This operation may occur on a server in the doctor's office or may occur on a remote server, or may occur on the user's computing device or on the vaporizer itself, in embodiments in which the vaporizer has the appropriate computing chips and software installed.

If the material in the installed cartridge matches the material prescribed in the prescription, then the vaporizing device will be unlocked or otherwise activated for operation. If the material does not match, the vaporizer will remain locked. Optionally, in this embodiment, the vaporizing device operation also requires authentication of the user before operation of the device is permitted. This may be accomplished by requiring password entry. Passwords may be generated by the prescribing physician or may be selected by the end user themselves. In cases where the doctor or doctor's server issues a password, this operation may be used to ensure that the end user is authorized to operate the vaporizer device (and may prevent operation by others if lost and then found by others for example). In embodiments in which the password or similar security feature is selected or enabled by the end user, this feature may be utilized to prevent children in a household from operating the device (for example). Of course, multiple passwords for multiple layers of security may be employed, including doctor issued passcodes combined with user selected passcodes.

In preferred, but still optional, embodiments, the user can select a burn or vaporizing temperature of the material to be vaporized. This can be done for personal preference of to avoid overheating materials and creating carcinogens. Alternatively, the vaporizer device can automatically adjust the temperature of the heating element based on stored data associated with the vaporizable material detected in the cartridge. This data can also be generated from either user preference or for safety or health reasons.

Once the vaporizer device is cleared for usage after material verification and after any optional security or authentication steps are performed, the device may be operated. During operation, the heating element of the vaporizer device is activated to heat the vaporizable material so that it turns into a gas and may be ingested by the end user. When the gas is ingested, the volume of gas ingested with each inhalation is preferable measured using a volume flow meter and recorded and/or transmitted to the network or to the accompanying computing device. The volume of gas ingested, as detected by the volume flow meter, is then compared to the prescription issued by the doctor (preferably automatically by operation of the configured software and hardware). Continued use of the vaporizer device is permitted until the limits of the prescribed dosage amounts are reached. Once the dosage limit is reached, the device automatically disables itself so that no more prescribed material may be ingested.

Similarly, a prescription may include dosage times. In at least one of such embodiments, material may only be vaporized for ingestion at times selected by the prescribing doctor. For example, if the prescribing doctor prescribes the material to assist in sleep, the prescribing doctor can issue a prescription, which is stored within the network to be read and accessed by the vaporizer device, so that the vaporizing device may only be used during the hours prior to conventional sleep times. In such an embodiment, operation during morning hours would not be permitted, and the vaporizing device would simply remain deactivated during non-prescribed hours.

Once the security or authentication protocols are passed, the user may then operate the device and ingest vaporized material. In preferred embodiments, upon each inhalation of each vaporizer dose, the vaporizer communicates a signal to a computer processing device, which then prompts the user of the device to provide feedback on his/her experience with the prescribed material. For example, if upon inhalation, the user thereafter feels relaxed, the user can provide data input to communicate this achieve result. Similarly, if the inhaled dose makes the user feel excited, the user can input data indicating this physical condition. In preferred embodiments, the user is prompted to enter usage results data directly into a graphical user interface. In more preferred embodiments, the user interface provides a pre-selected number of conditions to choose from, so that the user can quickly enter results data. A non-exhaustive list of material inhalation effects or results, to be presented to a vaporizer device user (for selection) are: satisfaction, dissatisfaction, positive effect, negative effect, happy, sad, relaxed, agitated, energetic, inactive, social, anti-social, focused, scattered, motivated, discouraged, and no effect.

Once the effect or result experienced upon inhalation is entered into the computing device, the experience data is recorded or stored and/or transmitted through the network to a data collection unit or to the prescribing physician. In the latter example, the data can be collected by a computer system of the prescribing physician so that the physician can monitor the effects of the prescribed material to ensure that the prescription is effective, for example. In other embodiments, the data of many users (e.g., thousands of users) is collected and then a database is assimilated based on collected data, so that the true therapeutic or medical effects of a prescribed vaporizable material can be known. In similar embodiments, the device can be used as part of a clinical trial, and the data input by the end device users can be transmitted to a data collection center for analysis by the entity administering a drug trial.

Once given the above disclosure, many other features, modifications, and improvements will become apparent to the skilled artisan. Such features, modifications, and improvements are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally set forth as limiting, is not intended to impart a narrowing scope to the invention in contravention of the ordinary meaning of the claim terms by which the scope of the patent property rights shall otherwise be determined:

We claim:

1. A method for monitoring and regulating a user's consumption of vaporizing materials comprising:
providing a vaporizing device including at least one flow meter and heating element associated therewith;
providing at least one computer processor and at least one computer readable medium in communication with said computer processor; the computer readable medium and the computer processor being communicably connected to a network; the computer readable medium including, stored thereon, a set of computer readable and executable instructions for processing by the computer processor, the computer readable and executable instructions being defined to permit control of the parameters of vaporizer usage and being defined to permit data monitoring of said vaporizer usage; the computer readable and executable instructions also defining a vaporizer end user interface, comprised of graphical user interface components;
providing one or more data input devices configured to permit one or more users to input data, in the form of transmittable data, corresponding to vaporizing device usage, for storage, for analysis, or for transmission to a monitoring entity;
providing one or more display screens configured for displaying said graphical user interface and for displaying vaporizer data and;
configuring said flow meter so that when said vaporizing device is operated by an end user to ingest vaporized gas, the volume of ingested vaporized gas is measured and a data point is collected and processed by said computer processor; and
configuring said graphical user interface to prompt the end user to enter data pertaining to the end user's experience upon ingestion of said vaporized gas.

2. The method according to claim 1 further including the following method steps:
defining a vaporizing gas dosage determined as having characteristics and qualities beneficial to an end user, said dosage comprising a volume of gas having a quantity X;
using said flow meter to measure a first volume of vaporized gas ingested by the end user, as said vaporized gas passes through said flow meter, and assigning said first volume of vaporized gas ingested as quantity Y₁;
comparing the value of quantity X to the value of quantity Y₁ and if the value of quantity Y₁ is less than the value of quantity X, said computer processer processing said computer readable and executable instructions permits the continued operation of said vaporizing device, but if the value of quantity Y₁ is approximately equal to or greater than the value of quantity X, said computer processer processing said computer readable and executable instructions at least temporarily shuts down vaporizing functions of said vaporizing device so that the end user is prevented from ingesting further quantities of vaporized gas.

3. The method according to claim 2 further including the following method steps:
using said flow meter to measure a second volume of vaporized gas ingested by the end user, as said vaporized gas passes through said flow meter, and assigning said second volume of vaporized gas ingested as quantity Y₂;
said computer processer processing said computer readable and executable instructions automatically adding the value of quantity of Y₁ to the value of quantity Y₂ to obtain a sum representing the cumulative volume Y_{C} of vaporized gas ingested by an end user;
comparing the value of quantity X to the value of cumulative volume Y_{C} and if the value of cumulative volume Y_{C} is less than the value of quantity X, said computer processer processing said computer readable and executable instructions permits the continued operation of said vaporizing device, but if the value of cumulative volume Y_{C} is approximately equal to or greater than the value of quantity X, said computer processer processing said computer readable and executable instructions at least temporarily shuts down vaporizing functions of said vaporizing device so that the end user is prevented from ingesting further quantities of vaporized gas.

4. The method according to claim 3 further including the following method steps:
said monitoring entity, and not the end user, selecting said vaporizing gas dosage having a quantity X and transmitting information corresponding to said vaporizing gas dosage from a monitoring entity location to a location corresponding to said data input device and said vaporizing device;
wherein because said monitoring entity selects said vaporizing gas dosage having a quantity X, said monitoring entity maintains restrictive control of the type and amount of vaporized gas ingested by the end user.

5. The method according to claim 4 further wherein said monitoring entity is a physician and said vaporizing gas dosage having a quantity X is selected and designated pursuant to a prescription prescribing a quantity of ingestible material, and wherein said vaporizing gas dosage having a quantity X is transmitted to an end user patient of said licensed physician over communication network such that data corresponding to said vaporizing gas dosage having a quantity X is received by said data input device and said vaporizing device of the end user patient; and
wherein said computer processer processing said computer readable and executable instructions controls the operating status of said vaporizing device so that the end user is only permitted to ingest vaporizing material utilizing said vaporizing device in accordance with said prescription.

6. The method according to claim 1 further including the following method steps:
provisioning said set of computer readable and executable instructions for processing by the computer processor to include a security protocol;
provisioning said data input device to require compliance with said security protocol prior to said computer processor activating said vaporizing device for usage by an end user.

7. The method according to claim 5 further including the following method steps:
provisioning said set of computer readable and executable instructions for processing by the computer processor to include a security protocol;
provisioning said data input device to require compliance with said security protocol prior to said computer processor activating said vaporizing device for usage by an end user.

8. The method according to claim 6 wherein said security protocol is a password requirement and wherein a password must be entered into said data input device utilizing said graphical user interface; and
wherein upon successful entry of a correct password into said data input device, said computer processor activates said vaporizing device for usage by an end user.

9. The method according to claim 7 wherein said security protocol is a password requirement and wherein a password must be entered into said data input device utilizing said graphical user interface; and
wherein upon successful entry of a correct password into said data input device, said computer processor activates said vaporizing device for usage by an end user.

10. The method according to claim 8 further including the following method steps:
provisioning said set of computer readable and executable instructions for processing by the computer processor so that said security protocol automatically deactivates said vaporizing device after a predetermined duration of non-usage of said vaporizing device is measured.

11. The method according to claim 9 further including the following method steps:
provisioning said set of computer readable and executable instructions for processing by the computer processor so that said security protocol automatically deactivates said vaporizing device after a predetermined duration of non-usage of said vaporizing device is measured.

12. The method according to claim 5 further including the following method steps:
provisioning said set of computer readable and executable instructions for processing by the computer processor to include a security protocol;
provisioning said data input device to require compliance with said security protocol prior to said computer processor activating said vaporizing device for usage by an end user; and
wherein said security protocol comprises automatically machine determining the identity of the ingestible material installed in said vaporizing device and said computer processor comparing said identity of said ingestible material to data contained in said prescription of said physician;
wherein if said identity of said ingestible material matches data in said prescription of said physician, said vaporizing device is activated for usage by said computer processor; and
wherein if said identity of said ingestible material does not match data in said prescription of said physician, said vaporizing device remains deactivated and cannot be used to vaporize material for ingestion by an end user.

13. The method according to claim 9 further including the following method steps:
provisioning said set of computer readable and executable instructions for processing by the computer processor to include a security protocol;
provisioning said data input device to require compliance with said security protocol prior to said computer processor activating said vaporizing device for usage by an end user; and
wherein said security protocol comprises automatically machine determining the identity of the ingestible material installed in said vaporizing device and said computer processor comparing said identity of said ingestible material to data contained in said prescription of said physician;
wherein if said identity of said ingestible material matches data in said prescription of said physician, said vaporizing device is activated for usage by said computer processor; and
wherein if said identity of said ingestible material does not match data in said prescription of said physician, said vaporizing device remains deactivated and cannot be used to vaporize material for ingestion by an end user.

14. The method according to claim 12 further wherein said monitoring entity is an entity collecting data for use and analysis in clinical trials; and
wherein said input data, input by users, corresponding to vaporizing device usage, and pertaining to the end user's experience upon ingestion of said vaporized gas, is collected and analyzed in connection with a clinical trial associated with said ingestion of said vaporized gas.

15. The method according to claim 14 wherein said method step of prompting the end user to enter data pertaining to the end user's experience upon ingestion of said vaporized gas includes prompting the end user to enter one or more data points selected from the group consisting of: satisfaction, dissatisfaction, positive effect, negative effect, happy, sad, relaxed, agitated, energetic, inactive, social, anti-social, focused, scattered, motivated, discouraged, and no effect.

16. The method according to claim 1 wherein said method step of prompting the end user to enter data pertaining to the end user's experience upon ingestion of said vaporized gas includes prompting the end user to enter one or more data points selected from the group consisting of:
satisfaction, dissatisfaction, positive effect, negative effect, happy, sad, relaxed, agitated, energetic, inactive, social, anti-social, focused, scattered, motivated, discouraged, and no effect.

17. A method for monitoring and regulating a user's consumption of vaporizing materials comprising:
providing a vaporizing device including at least one flow meter and heating element associated therewith;
providing at least one computer processor and at least one computer readable medium in communication with said computer processor; the computer readable medium and the computer processor being communicably connected to a network; the computer readable medium including, stored thereon, a set of computer readable and executable instructions for processing by the computer processor, the computer readable and executable instructions being defined to permit control of the parameters of vaporizer usage and being defined to permit data monitoring of said vaporizer usage; the computer readable and executable instructions also defining a vaporizer end user interface, comprised of graphical user interface components;
providing one or more data input devices configured to permit one or more users to input data, in the form of transmittable data, corresponding to vaporizing device usage, for storage, for analysis, or for transmission to a monitoring entity;
providing one or more display screens configured for displaying said graphical user interface and for displaying vaporizer data and;
configuring said flow meter so that when said vaporizing device is operated by an end user to ingest vaporized gas, the volume of ingested vaporized gas is measured and a data point is collected and processed by said computer processor;
configuring said graphical user interface to prompt the end user to enter data pertaining to the end user's experience upon ingestion of said vaporized gas; and
automatically machine determining the identity of the ingestible material installed in said vaporizing device;
installing a quantity of vaporizable and ingestible material at an intake portion of said vaporizing device; and
automatically machine determining the identity of said vaporizable and ingestible material.

18. The method according to claim 17 further including the following method step:
selecting a temperature of said heating element as a vaporizing temperature of said vaporizable and ingestible material.

19. The method according to claim 18 further including the following method step:
said computer processor comparing said identity of said vaporizable and ingestible material to data stored pertaining to said vaporizable and ingestible material; and
automatically machine determining said temperature of said heating element as a vaporizing temperature of said vaporizable and ingestible material, using said data stored pertaining to said vaporizable and ingestible material.

20. The method according to claim 19 where said step of installing said quantity of vaporizable and ingestible material at said intake portion of said vaporizing device is performed by installing a removable cartridge containing said vaporizable and ingestible material to said intake portion of said vaporizing device.

21. The method according to claim 20 further wherein said removable cartridge includes data information pertaining to the identity of said vaporizable and ingestible material and wherein said vaporizing device reads said data information in order to perform said step of automatically machine determining the identity of said vaporizable and ingestible material.
